(19) United States
(12) Patent Application Publication
(10) Pub. No.: US 2007/0043262 A1
(43) Pub. Date: Feb. 22, 2007

(54) Fluid supply for endoscope

(75) Inventors: Avi Levy, Herzylia (IL); Dan Oz, Even Yehuda (IL)

Correspondence Address: BRUCE E. LILLING;LILLING & LILLING P.C., P.O. BOX 560, GOLDEN BRIDGE, NY, 10526, US

(73) Assignee: Sightline Technologies Ltd., Haifa (IL)

(21) Appl. No.: 11/207,093

(22) Filed: Aug. 18, 2005

(51) Int. Cl. A61B 1/12

(52) U.S. Cl. 600/156; 600/158; 600/159

(57) ABSTRACT

An endoscopic apparatus and fluid supply unit is described. The endoscopic apparatus comprises an insertion member for insertion into said body lumen and having at least one channel through which a fluid medium is supplied to the body lumen. The endoscopic apparatus further comprises an operation handle, a control unit for controlling supply of the fluid medium, a fluid supply unit and a means for delivering said fluid medium from said fluid supply unit to said channel. The fluid supply unit is provided with at least one refillable or replaceable container for storing the fluid medium therein and for supplying the fluid medium to the channel upon receiving a signal from the control unit.

### FIELD OF THE INVENTION

The present invention relates generally to the field of endoscopy and specifically to fluid supply for endoscope apparatus used for endoscopic procedures during which a flexible or rigid tube is inserted into a body passage for examination of its interior for abnormalities.

### BACKGROUND OF THE INVENTION

There are known endoscopes employing at least one working channel extending along the insertion tube. Through this channel either vacuum can be supplied for suction from a body passage, or a surgical instrument can be inserted into the body channel or water can be supplied to the body channel via a Y-type port located just below the grip section of the operation handle. This port, which will be referred to further as the Y-port, is used for insertion and evacuation therethrough of a surgical instrument. An example of such an endoscope can be found in Furuya (U.S. Pat. No. 6,881,188).

During the endoscopic procedure it is often required to wash the body channel and/or to supply thereto various liquids, e.g. saline, medicine, liquids for providing contrast etc. Unfortunately the existing irrigation arrangement, which is intended mainly to clean the optical window, is not well suited for this purpose, since it does not allow supplying fluids with a controllable feed rate, which might be required for proper washing of the body channel.

In practice a syringe filled with the required liquid is used for washing the body channel and for introducing therein other liquids. The syringe is connected to the working channel through a port provided at the operation handle of the endoscope and then the washing liquid or other liquid is manually pumped (by doctor's or assistant's hand) from the syringe to the working channel and to the body lumen. Examples of syringe liquid supply can be found in Brown (U.S. Pat. No. 5,167,220), Harhen (U.S. Pat. No. 6,190,330), JP 9313431, JP 2000237132. The disadvantage of this supply is associated with the necessity to use several syringes, and each of them has to be periodically connected and disconnected from the working channel. The liquid supply is carried out before or during the endoscopic procedure. It can be readily appreciated that each time the syringe is in use it is not possible to use the surgical tool, since the syringe occupies the port and the surgical tool cannot be inserted thereinto. Furthermore, since during actuation of the syringe at least one of the doctor's hands is occupied, the operation of the endoscope becomes inconvenient and might necessitate assistance of a nurse. Still further disadvantage of the syringe liquid supply is associated with the difficulty to control the feed rate during manipulation of the syringe.

Another disadvantage of the syringe fluid supply is associated with the impossibility to mix two or more liquids from one or more syringes.

Still further disadvantage lies in the fact that the amount of liquid, which can be supplied by a syringe, is limited.

There exist also more sophisticated solutions for using the syringe liquid supply. For example in CA 2114,018 is disclosed an actuator for actuating an irrigation syringe. The actuator includes a food pedal assembly, which actuates the syringe and thus frees the hands of the operator. Unfortunately this device is rather complicated, still requires replacement of syringes and suffers from the other drawbacks, which have been mentioned above.

There are also known endoscopes, which employ a single integral pipe instead of separate working channels. This pipe is also known as multilumen tubing, since it is usually fitted with appropriate passages or lumens extending along the pipe and intended for irrigation, for insufflation, for suction and for passing endoscopic tools therethrough. An example of such an endoscope can be found in Eizenfeld (WO 2004/016299) which is incorporated herein by reference. For operating the endoscope, which is disclosed in this reference, the proximal end of the multilumen tubing is detachably connected through a dedicated connector to respective tubes or hoses, which supply water and air from a control unit and vacuum from a vacuum source. Furthermore, the connection also has a dedicated instrument channel port arranged in a Y-type fashion just below the grip section of the operation handle.

In our International patent application PCT/IL2005/000428 a connector is described for use with the multilumen tubing. This connector, which will be further referred to as the Y-connector, is removably attachable to the Y-port and serves for establishing flow communication between the passages of the multilumen tubing and between the tubes for supplying fluid medium and vacuum for suction from the control unit to the multilumen tubing. The Y-connector is also adapted for insertion and retraction of a surgical instrument therethrough. The Y-connector comprises a main body portion provided with a through-going axial bore and a plurality of lateral ports, which are in flow communication with the said bore irrespective to the axial angle at which said Y-connector is attached to the Y-port. The axial bore permits insertion thereinto and removal therefrom of a proximal end of the multilumen tubing and the lateral ports receive thereinto the tubes for supplying the fluid medium.

There also exist alternative solutions for liquid supply, which do not employ syringes.

For example in Desai (U.S. Pat. No. 6,375,653) a fluid supply for a rigid endoscopic instrument is described. This fluid supply comprises an irrigation pump connected to a source of liquid and to an irrigation port of the endoscope.

In JP 2001292963 a device for supplying water to an endoscope is described. This device also employs a pump, which forcibly supplies water from a dedicated container to the endoscope for washing the body cavity.

More or less similar solutions can be found in JP 2003045779, JP 20001139825, JP 2002301013, JP 2002085340, JP 2003032862, JP 5049595.

It should be kept in mind, however, that the above-mentioned solutions are based on supply of a fluid from a container to a port, which is located in the upper part of the operation handle and not to the Y-port at the operation handle.

It would be very desirable to replace the existing syringe liquid supply by a new fluid supply, which would be connectable to the Y-port of an endoscope.

### SUMMARY OF THE INVENTION

The object of the present invention is to provide a new and improved fluid supply for supplying fluid medium to the working channel of an endoscopic apparatus.

A further object of the invention is to provide a new fluid supply, which eliminates the necessity of a syringe.

Still a further object of the invention is to provide a new fluid supply, which has very simple construction, is convenient in use and does not occupy the doctor's hands.

Another object of the invention is to provide a new fluid supply, which is suitable for supplying various fluids to the working channel of an endoscopic apparatus via the Y-port at the operating handle.

An additional object of the invention is to provide a new fluid supply, which allows mixing of various fluids to be supplied to the working channel of an endoscopic apparatus.

Still a further object of the invention is to provide a new fluid supply, which allows supply of various fluids to the working channel of an endoscopic apparatus irrespective of whether the surgical tool is located or not in the channel.

A further object of the invention is to provide a new fluid supply, which controls the feed rate and pressure of the fluids, which are being supplied to the working channel of an endoscopic apparatus.

Yet another object of the invention is to provide a new and improved fluid supply, which would be connectable to either the axial port or to the lateral ports of the existing Y-connectors.

For a better understanding of the present invention as well of its benefits and advantages, reference will now be made to the following description of its embodiments taken in combination with the accompanying drawings.

### BRIEF DESCRIPTION OF THE DRAWINGS

FIG. 1 schematically depicts the prior art solution employing a syringe.

FIG. 2 depicts a general view of an endoscopic apparatus, preferably a colonoscopic apparatus, provided with the fluid supply of the invention.

FIGS. 3a and 3b show schematically how the fluid supply depicted in FIG. 2 can be connected to the source of pressure and to the operation handle.

FIG. 4 is a cross-sectional view of the Y-connector used with the fluid supply of the invention.

FIG. 5a and FIG. 5b are end views of the Y-connector of FIG. 4 and show schematically how sealing is achieved in the axial port of the Y-connector when a surgical instrument is inserted.

FIG. 6a shows an alternate embodiment of the invention, in which the fluid supply is connected laterally to a single axial port of the Y-connector.

FIG. 6b is a schematic view if the fluid supply unit for the embodiment shown in FIG. 6a.

FIG. 7 is a schematic view of an auxiliary fitting member used in the embodiment shown in FIG. 6.

FIG. 8 is a schematic view showing the common supply tube directly connected to the Y-connector.

### DETAILED DESCRIPTION OF THE INVENTION

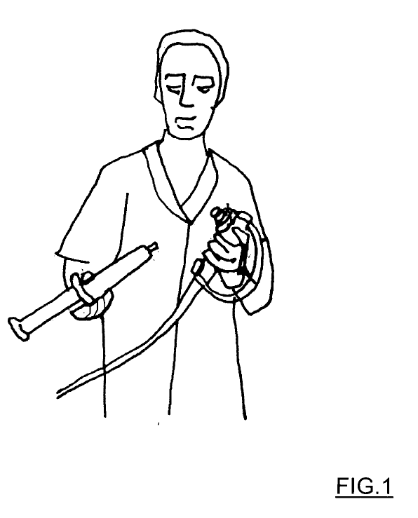

With reference to FIG. 1, a prior art solution is shown used for the supply of washing liquid to a colonoscope. In accordance with this solution, the doctor uses a syringe connectable to the operating handle. One can readily appreciate that for actuating the syringe only one of the doctor's hand is available, since the other hand is used for actuating the operation handle. Due to this reason it is not convenient to operate the syringe.

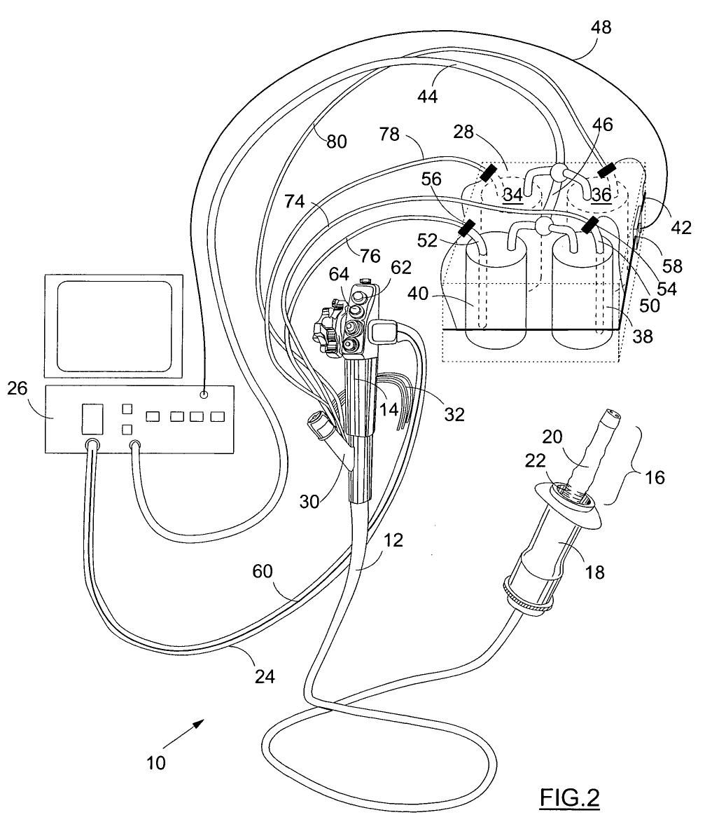

In FIG. 2 an embodiment of the fluid supply of the present invention is shown when it is used with a colonoscopic apparatus 10, which is provided with inflatable propelling sleeve. It should be appreciated, however, that this is only an option, since the new fluid supply is suitable for any colonoscopic or endoscopic apparatus, irrespective of whether it employs or not the propelling sleeve. The colonoscopic apparatus shown in FIG. 2 comprises an endoscope portion with an insertion tube, which proximal section 12 is connected to an operation handle 14 and its distal section 16 is inserted in and protrudes from a disposable dispenser 18, in which resides the inflatable disposable propelling sleeve.

It is seen also in FIG. 2 that the sleeve covers the distal section of the endoscope. That part of the inflatable propelling sleeve, which is seen in FIG. 2, comprises a frontal non-inflatable portion 20 and a rear, folded portion 22. The frontal portion of the sleeve covers the distal section of the endoscope and its head. The frontal portion does not inflate when the endoscope advances within the colon. The rear portion covers the insertion tube and unfolds when air or other fluid medium is supplied to the sleeve. By virtue of this provision the endoscope is pushed forward within the body passage while the sleeve feeds out from the dispenser. Explanation of this phenomenon can be found in Eizenfeld (WO 2004/016299). The endoscope shown in FIG. 2 is of similar type in the sense that it employs the same propelling mechanism, which is based on inflation of the flexible disposable sleeve coupled to the endoscope. It should be appreciated, however, that the present invention is not limited merely to a propelling colonoscope in particular or colonoscopy in general. It can be employed in any other medical procedure requiring insertion of a probe in a body passage for inspection of its interior.

It is seen also in FIG. 2, that the handle is connected by an umbilical cord 24 to a control unit 26 having a source of compressed air for inflating and venting the inflatable propelling sleeve. The control unit can be also provided with a flask (not shown) filled with water used either for irrigation of the optical lens at the forwardmost end of the of the insertion tube or for supplying to the body lumen.

Within the insertion tube are provided various devices, which are necessary for proper functioning of the endoscope. These devices are known per se. Among such devices one can mention vertebrae and strings, which can be manipulated by angulation control knobs provided on the operating handle. Another device is a multilumen tubing extending along the insertion tube and provided with appropriate passages or lumens for supplying air for insufflation, water for irrigation and vacuum for suction. The suction channel is used also for introducing surgical instruments as might be required during the endoscopic procedure.

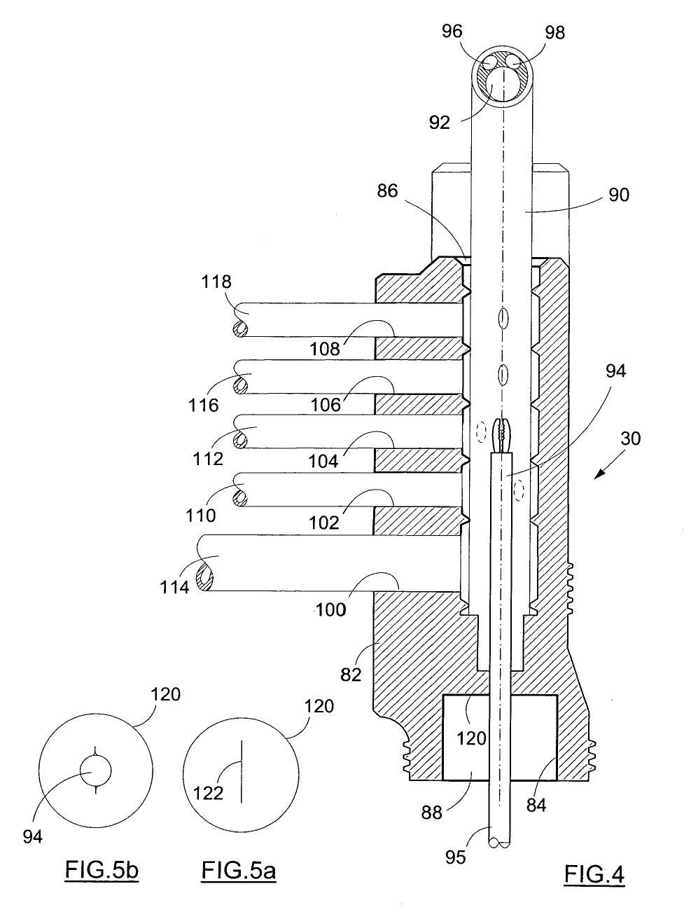

It should be kept in mind that instead of the integral multilumen tubing one could use separate channels. The multilumen tubing will be explained in more detail hereinafter with reference to FIG. 4.

Still referring to FIG. 2, the multilumen tubing extends through the insertion tube and through the lower part of the operation handle to a Y-connector 30, which has lateral ports and an axial port. The lateral ports are used for flow communication between channels of the multilumen tubing and hoses 32 passing along the umbilical cord. These hoses are used for supplying insufflation air from the control unit, irrigation water from the flask and vacuum from a vacuum source. The Y-connector 30 will be explained in more detail hereinafter with reference to FIG. 4

In accordance with the present invention in addition to the existing irrigation arrangement, the endoscopic apparatus is provided with a fluid supply unit 28, which is pneumatically and electrically connected to the control unit and hydraulically connected to the Y-connector 30. This fluid supply unit can be internal or external with respect to the control unit 26. The fluid supply unit is intended for supplying one or more different fluids to the body lumen. The liquids are supplied from dedicated refillable or replaceable containers residing within the fluid supply unit. The non-limited list of fluids comprises cleaning liquids, medicines, liquids for obtaining contrast, oils etc. The fluid supply unit can supply water and/or other liquids for washing or other purposes to the body lumen. The fluid supply unit will be described in more detail hereinafter.

Referring again to FIG. 2 it is seen that the fluid supply unit 28 is provided with four containers 34, 36, 38, 40, which reside within a common housing 42. The containers are deployed within the unit with possibility for replacement or refill. As stated above, the containers are filled with different fluids, which might be required during the endoscopic procedure. It should be kept in mind that the amount of containers can vary depending on the amount of fluids required for the endoscopic procedure. In some cases it might be sufficient to use only one container, which is filled with water supplied immediately to the body lumen for its washing.

A common duct 44 is provided, which supplies air pressure to the containers 34, 36, 38, 40 from a pump (not shown) installed in the control unit 26. In addition to or instead of this arrangement the pump could be separate from the control unit or be part of the fluid supply unit. Furthermore, instead of the common duct for supplying pressure to the containers one could use separate ducts connected to each container. All these arrangements should be suitable for supplying a fluid to the Y-connector 30 either from one container at a time or simultaneously from more then one container, thus providing a possibility for mixing fluids.

The common duct terminates within the housing 42 by a manifold 46, through which air pressure is permanently supplied to the containers 34, 36, 38, 40. By virtue of this provision the fluids are permanently kept in a pressurized condition in the containers.

Each container is provided with an exit port, which is normally closed by a pinch valve or by any other electrically controllable valve. In FIG. 2 are seen only exit ports 50, 52 referring to containers 38, 40. These ports are shown by solid lines. Respective valves 54,56 normally close these ports. Respective fluid supply tubes connect each exit port to a corresponding lateral port provided on the Y-connector 30. In FIG. 2 are seen four fluid supply tubes 74, 76, 78, 80 connected respectively to four containers 38, 40, 34, 36. It is seen in FIG. 2 that a first cable 60 is provided. The first cable extends along the umbilical cord and electrically connects the control unit 26 and the electrical control buttons 62, 64 provided at the operation handle 14. The number of control buttons corresponds to the amount of containers. By pushing a button the doctor generates an electrical control signal, which is transmitted to the control unit 26. The signal is then transmitted by the control unit via the second cable 48 and electrical connector 58, situated on one of the walls of the housing 42, to the fluid supply unit 28 and the valve of that respective container, whose control button has been pushed. According to the control signal the valve will be opened or kept closed. For the sake of brevity only two control buttons 62, 64 are shown in FIG. 2. These buttons control valves 54, 56 that open respective exit ports 50, 52 and allow the liquids to exit from the respective containers 38, 40. It can be readily appreciated that the fluid will be forced by pressure to exit from the container as soon as its valve receives the electrical control signal to change the normally closed condition to an open condition. The fluid exits from the open container and proceeds through the respective supply tube to one of the lateral ports of the Y-connector 30 and then via the multilumen tubing to the body lumen.

Thus, the main idea of the present invention is to provide the existing endoscopic apparatus with a new fluid supply, which comprises one or a plurality of replaceable containers filled with various fluids as might be required during the endoscopic procedure and in particular for washing the colon. Each container can be refilled or replaced when it becomes empty and therefore the fluid supply unit, in fact, comprises a dispenser fitted with replaceable containers filled with the fluids.

The fluids are kept in the containers and can be forcibly supplied by pressure to the body lumen selectively and in a controllable fashion through the existing Y-port of the operating handle.

It can be readily appreciated that the pressure in the container can be varied and therefore the liquid can be supplied to the body channel with a controllable feed rate. Alternatively, the valves can be of a variable type or flow regulator type, hence the liquid can be supplied to the body channel with a controllable feed rate. By virtue of this provision it is possible to improve the efficiency of washing or irrigation.

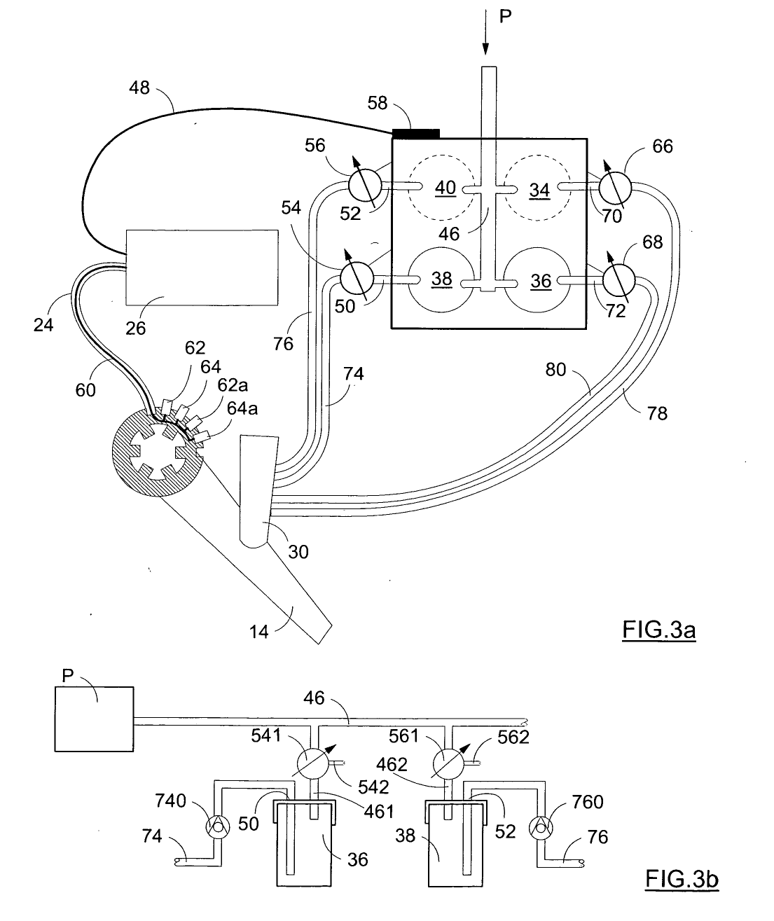

With reference to FIG. 3a an embodiment of the present invention is shown in a more schematic way. Similar reference numerals are used for designating similar elements of the colonoscopic apparatus and of the fluid supply unit. Four valves 54, 56, 66, 68 are seen, which upon pressing buttons 62, 64, 62a, 64a on the operating handle 14 control the exit of liquids from containers 38, 40, 34, 36, through respective exit ports 50, 52, 70, 72 via supply tubes 74, 76, 78, 80 to lateral ports of Y-connector 30. The fluids in the containers are kept under pressure P supplied to the manifold 46 from an external source or from the control unit 26.

In the embodiment shown in FIG. 3a the fluids are kept in containers under permanent pressure and each container is fitted with a dedicated valve located at the exit from each container.

Referring now to FIG. 3b there is schematically shown another embodiment of the fluid supply. For the sake of brevity only two containers 36, 38 are shown, which are connected by the common manifold 46 to the source of pressure P. Each container is hermetically closed by a closure through which passes a respective branch 461, 462 of the manifold. Respective exit ports 50,52 are seen being arranged in respective closures. It should be appreciated that a similar arrangement is provided in the embodiment shown in FIG. 3a.

In contrast to the previous embodiment, in the embodiment depicted in FIG. 3b each container is provided with respective valves 541, 561, which are located at the entrance of each container. By virtue of this provision the fluid is not kept under permanent pressure and is forcibly released only when the valve is open to admit the pressure into the container. It would be advantageous if the valves were provided with relief openings 542, 562 connected to atmosphere so as to release pressure from the manifold when no flow is needed.

Furthermore, it would be also beneficial if after exit ports 50, 52 respective check valves 740, 760 were provided before supply tubes 74, 76 to prevent back flow of fluids.

In addition, appropriate valves (not shown) should be added for prevention of liquids in the containers flowing out in case the operator applies suction to the working channel of the multilumen tubing.

Various types of valves can be employed either at the entrance or the exit of the containers, which are capable of varying the flow rate e.g. on/off valves or flow regulator valves.

Referring now to FIG. 4 an example of the Y-connector 30 for use with the fluid supply unit 28 of the present invention will be explained.

In principle this Y-connector can be similar to the connector described in our patent application PCT/IL2005/000428. This Y-connector comprises a main body portion 82 with a proximal port 84. The connector is detachably connectable to the operation handle 14. Within the main body portion 82, a longitudinally extending through-going bore is provided which extends between an exit opening 86 and an entry opening 88. This through going bore is intended for receiving of the multilumen tubing 90, whose proximal end is insertable through the exit opening 86. The inside diameter of the through going bore is selected in such a manner, that it is more than the outside diameter of the multilumen tubing so as to allow insertion of the multilumen tubing into the connector. Along the multilumen tubing extends a channel 92 through which a surgical instrument 94 can be inserted. This channel serves also for suction. Two more channels 96, 98 are provided, through which insufflation air and irrigation water are supplied. Directed transversally with respect to the bore three main lateral ports 100, 102, 104 and two auxiliary lateral ports 106, 108 are shown. All lateral ports are in fluid communication with the respective channels of the multilumen tubing by virtue of tangential windows, which are cut in the peripheral wall of the multilumen tubing. An explanation of this design can be found in our patent application PCT/IL2005/000428, which is incorporated herein by reference.

One should keep in mind that the fluid supply unit 28 of the present invention is not limited to using strictly with the Y-connector provided with tangential windows for connecting to channels of the multilumen tubing. The fluid supply unit of the present invention can be employed with any other Y-connectors, including those, which are provided with a separate working channel instead of the multilumen tubing.

Two main lateral ports 102, 104 are intended for connecting to supply tubes 110, 112, which respectively supply irrigation water and insufflation air to channels 96, 98 of the multilumen tubing. The third main lateral port 100 is intended for connecting to a fluid supply tube 114 through which vacuum is supplied to channel 92. In practice ports 110, 112 are of similar diameter, which is less than the diameter of port 100.

In conventional endoscopic apparatus the fluid supply tubes are connectable to the sources of water and air provided in the control unit 26 and to a vacuum source. In the fluid supply unit 28 of the present invention there are provided additional fluid supply tubes, which are connected to auxiliary lateral ports of the Y-connector. Through these tubes fluids from containers of the fluid supply unit are supplied to the Y-connector. The fluids are supplied through auxiliary lateral ports 106, 108 to channel 92 of the multilumen tubing. For the sake of simplicity the Y-connector is depicted with only two auxiliary lateral ports 106, 108 as would be required for the fluid supply unit provided with two containers. It should be born in mind however, that for the fluid supply unit provided with four containers more lateral ports would be required to accommodate the fluid supply tubes. This situation is depicted in FIG. 2, in which are seen three main lateral ports collectively designated by reference numeral 32. These ports are in fluid communication with the control unit 26. There are also seen four auxiliary lateral ports, which are in fluid communication with the containers of the fluid supply unit.

Referring back to FIG. 4 and to FIGS. 5a and 5b, a partition 120 is seen, which can be integral with or separate from the main body portion. The partition as well as the main body portion are made of a resilient material, e.g. polyurethane and by virtue of this provision the partition creates a seal between the through going bore and the entry opening for a surgical tool and prevents flow communication between the through going bore and the open atmosphere when vacuum or liquid is supplied to the channel 92.

It is schematically shown in FIG. 5a that a narrow slot 122 is cut in the partition. Through this slot the surgical tool can be inserted within the Y-connector, while its shaft 94 remains sealed by resilient walls of the slot, as seen in FIG. 5b.

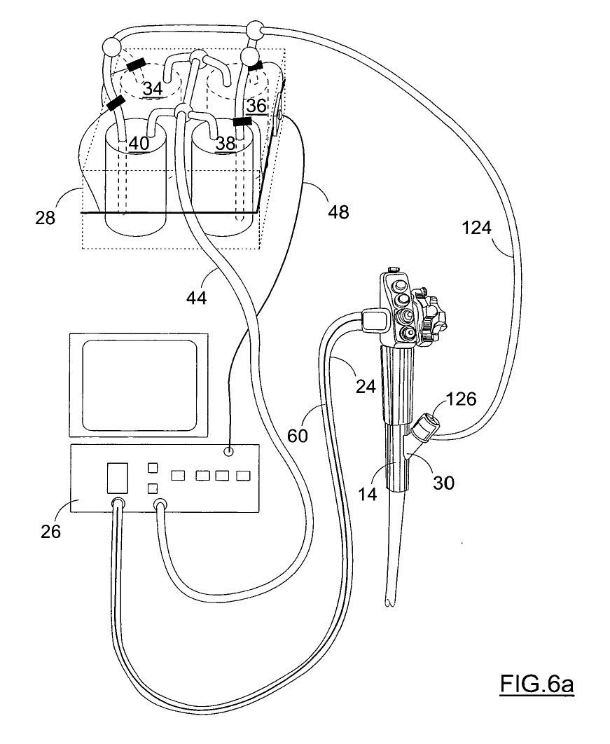

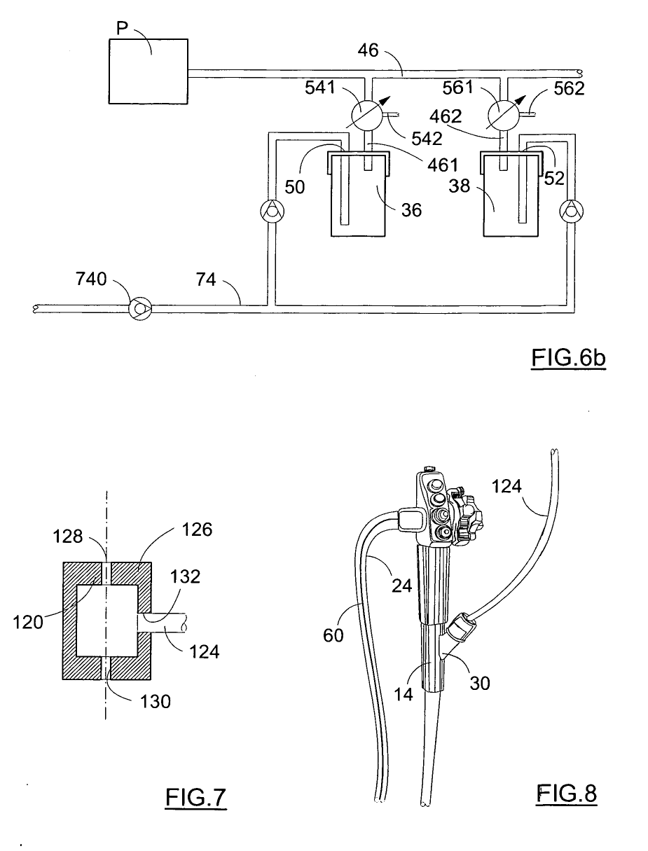

Now with reference to FIG. 6a a still further embodiment of the present invention will be explained. In this embodiment the endoscopic system is provided with similar components, which were already mentioned in connection with FIGS. 2, 3a, 3b and these components are designated in FIGS. 6a, 6b by the same reference numerals. However, in contrast to the previous embodiments the containers of the fluid supply unit are not connected to the respective separated supply tubes. Instead of this, all supply tubes are connected to a common supply tube 124, which is in fluid communication with the Y-connector 30 through a fitting member 126 connectable to the main body portion or made integral therewith. It is seen in FIG. 7, that the fitting member is provided with an axial entry port 128, an axial exit port 130 and a lateral port 132. The common supply tube 124 is connected to the lateral port 132. Supplying of various fluids from containers of the fluid supply unit to the working channel 92 of the multilumen tubing is possible via axial exit port 130.

For a standard endoscope fitted with separate channels for air insufflation, water irrigation and working channel, the Y-connector will be provided with the fitting member fitted with only one lateral port 132 for receiving supply tube 124. The fluid communication with the working channel will be possible without occupying the axial entry port 128 and therefore a surgical instrument can be inserted into the working channel while fluid is flowing into the channel.

In yet another embodiment shown in FIG. 6b, the control valves 541, 561 can be located in the air pressure input lines rather than at the exit ports of the containers.

In yet another embodiment, the supply tube 124 can be connected axially to the fitting member as seen in FIG. 8. In this embodiment the common supply tube will be brought in fluid communication with the working channel via axial entry port 128 and axial exit port 130.

Thus by virtue of the present invention various fluid medium can be selectively and automatically supplied to the body lumen via the existing Y-port of the operating handle.

In particular, by virtue of the present invention it is possible to carry out the washing of the body passage very effectively and conveniently and without using a syringe.

The fluid supply is carried out automatically, with controlled flow rate and pressure by virtue of a very simple and inexpensive fluid supply unit provided with replaceable containers/cartridges for storing the fluid medium.

It should be also appreciated that the invention is not limited to the above-described embodiments and that one ordinarily skilled in the art can make modifications without deviation from the scope of the invention, as will be defined in the appended claims.

For example the fluid supply unit can be used not only with flexible endoscopes but also with rigid endoscopes.

The fluid supply unit can be in fluid communication with the channels not via the Y-port of the operation handle, but via any other port provided thereon.

The control buttons can be either of on/off type, or linear buttons or their combination.

The control buttons can be provided at the control unit or at the fluid supply unit instead or in addition to the operation handle.

The control signal can be produced by foot pedals instead of control buttons or by a keyboard at the control unit or even by a dedicated voice actuation.

When used in the following claims, the meaning of terms “comprise”, “include”, “have” and their conjugates is “including but not limited to”.

It should also be appreciated that the features disclosed in the foregoing description, and/or in the following claims, and/or in the accompanying drawings may, both separately and in any combination thereof, be material for realizing the present invention in diverse forms thereof.

We claim:

1. An endoscopic apparatus for endoscopic investigation of a body lumen, said apparatus comprises
an insertion member for insertion into said body lumen and having at least one channel through which a fluid medium is supplied to the body lumen,
an operation handle to which the insertion member is connected and having at least one port through which the fluid medium is supplied to said channel,
a control unit for controlling supply of the fluid medium and connected to said operation handle,
a fluid supply unit having at least one refillable or replaceable container for storing the fluid medium therein and for supplying the fluid medium to the channel upon receiving a signal from the control unit, and
means for delivering said fluid medium from said fluid supply unit to said channel.

2. The endoscopic apparatus as defined in claim 1, in which said fluid supply unit comprises a plurality of containers.

3. The endoscopic apparatus as defined in claim 2, in which said containers are in fluid communication with at least one port provided at the operation handle.

4. The endoscopic apparatus as defined in claim 2, in which said fluid medium is supplied from the fluid supply unit by pressure admitted to the containers upon receiving the signal from the control unit.

5. The endoscopic apparatus as defined in claim 4, in which said operation handle is provided with at least one control button for producing the signal upon which the pressure is admitted to the at least one container.

6. The endoscopic apparatus as defined in claim 5, in which each container is provided with a respective valve controllable by the signal to admit pressure to the container or to cut pressure from the container upon receiving the signal.

7. The endoscopic apparatus as defined in claim 6, in which said valve is positioned at the entrance to the container.

8. The endoscopic apparatus as defined in claim 5, in which each container is kept under pressure.

9. The endoscopic apparatus as defined in claim 6, in which each container is provided with a respective valve controllable by the signal to allow liquid to flow out of the container or to prevent liquid from flowing out of the container.

10. The endoscopic apparatus as defined in claim 9, in which said valve is positioned at the exit from the container.

11. The endoscopic apparatus as defined in claim 4, in which said containers are in fluid communication with a manifold connected to a source of pressure.

12. The endoscopic apparatus as defined in claim 4, in which said containers are provided with respective exit ports.

13. The endoscopic apparatus as defined in claim 12, in which said exit ports are connected to at least one port at operation handle.

14. The endoscopic apparatus as defined in claim 13, in which said exit ports are connected to the said at least one port at the operation handle through a common exit line.

15. A fluid supply unit for storing a fluid medium to be supplied to a body lumen via at least one channel extending in or along an insertion member of an endoscopic apparatus, wherein said fluid supply unit comprises:
a housing accommodating therein at least one replaceable or refillable container for storing the said fluid medium therein, said container being connected to a source of pressure and being provided with an entrance port and with an exit port,
a connector for electrical connection with a control unit of the endoscopic apparatus,
wherein said container is capable to release the fluid medium through the exit port upon receiving a signal from the control unit.

16. The fluid supply unit as defined in claim 15, comprising a plurality of containers.

17. The fluid supply unit as defined in claim 16, in which each container from said plurality is provided with a respective valve controllable by a signal to admit pressure to the container or to cut pressure from the container upon receiving the signal.

18. The fluid supply unit as defined in claim 17, in which said valve is positioned at the entrance to the container.

19. The fluid supply unit as defined in claim 16, in which each container from said plurality is provided with air pressure and a respective valve controllable by a signal to allow liquid to flow out the container or to prevent liquid from flowing out of the container.

20. The fluid supply unit as defined in claim 19, in which said valve is positioned at the exit from the container.

21. The fluid supply unit as defined in claim 15, in which at least one exit port is connected to a port provided at an operation handle of the endoscopic apparatus.

22. The fluid supply unit as defined in claim 16, in which exit port of each container from the plurality of containers is connected to a common exit line, which is in fluid communication with a port provided at an operation handle of the endoscopic apparatus.
